(19) United States
(12) Patent Application Publication
(10) Pub. No.: US 2015/0086609 A1
(43) Pub. Date: Mar. 26, 2015

(54) ADHESIVE LABEL WITH BITTERING AGENT AND FLUIDIFYING AGENTS FOR NATURAL AIRWAY SECRETTIONS

(71) Applicant: LABTEC GESELLSCHAFT FUR TECHNOLOGISCHE FORSCHUNG UND ENTWICKLUNG MBH, Langenfeld (DE)

(72) Inventor: Ingo Lehrke, Koln (DE)

(73) Assignee: LABTEC GESELLSCHAFT FUR TECHNOLOGISCHE FORSCHUNG UND ENTWICKLUNG MBH, Langenfeld (DE)

(21) Appl. No.: 14/558,459

(22) Filed: Dec. 2, 2014

Related U.S. Application Data
(63) Continuation of application No. 13/897,073, filed on May 17, 2013.
Application No. 13/897,073 is a continuation of application No. 12/445,147, filed on Feb. 1, 2010, now abandoned.
(60) Provisional application No. 60/852,475, filed on Oct. 17, 2006.

(51) Int. Cl. A61K 9/70; A61K 36/15; A61K 31/125; A61K 36/53; A61K 36/61; A61K 47/18

(52) U.S. Cl. CPC A61K 9/7084; A61K 36/61; A61K 47/186; A61K 31/125; A61K 36/53; A61K 36/15
USPC 424/443; 424/742

(57) ABSTRACT

Label for the outerwear for thinning of airway secretions, containing a bittering agent to reduce the likelihood of accidental ingestion.

### RELATIONSHIP TO PRIOR APPLICATIONS

This application claims priority to U.S. Provisional Application No. 60/852,475, filed Oct. 17, 2006.

### FIELD OF THE INVENTION

The present invention relates to patches or labels applied to outer garments that emit vapours for relieving congestion and other symptoms of the common cold.

### BACKGROUND OF THE INVENTION

Colds are a widespread ailment. They are characterized by the disagreeable result of congestion of the upper airway by a considerably increased amount of endogenous secretions, caused by the activity of viruses and/or bacteria. Thus, an immediate alleviation for the infected body exists, when draining of the secretions is facilitated.

It has long been known that the ingredients of plants that are rich with ether oils are suitable for this facilitation of draining. Such ether oils can be included as tea or in capsules (for example, Gelomyrtol™, Pohl-Boskamp), but often also cause irritation of the stomach and gall bladder. Therefore, often the topical use in the form of salves are used, which contain ether oils and which are to be applied by the patient on the chest, so that the substance penetrates the skin and enters the body via the airways. Corresponding preparations are Wick VapoRub™ (Wick Pharma), Bronchoforten™ (Plantorgan), and Pinimenthol™ (Spitzner), The duration of the substance release is limited to approximately one to two hours. A further problem of the salve application lies in the contamination of the hands with the slimy, skin-irritating ether oils. In order to avoid eye contact, it is essential that the patient washes his hands after use.

In order to avoid these disadvantages, carrier systems of non-woven material or fabric have been developed, which absorb the substance and enable a simpler application. These are either placed closed to the body (DE 4204222, DE 4007275, DE 3911617, DE 3823395) or adhered to the skin (DE 3540515) and then release the ether oils over a long time period on the skin side as well as into the atmosphere. In other developments, the material reservoir is enclosed between two films that are permeable to the material (DE 3902981, DE 3216609).

US Publication No. 20040156928 (Cordes et al.) describes an improvement over these earlier labels, in which the adhesive layer is dircetly adjacent the active ingredient layer, without any intervening protective layers. The label employs a specially formulated adhesive that resists solubilization by the active agents in the label.

The use of salves as well as the above-described systems bring the substances that are suitable for inhalation also in contact with the skin. The irritation of the skin by various ether oils (rosemary oil, turpentine oil, camphor) is known and is used for treatment of rheumatoid ailments. With the treatment of cold related illnesses with ether oils, this accompanying effect is not desired, however. But with common salve preparations and the above-described reservoir systems, this cannot be avoided.

Yet another problem with the foregoing delivery methods is the potential for poisoning when a child sucks on the label out of ignorance or abuse. U.S. Publication 20030064099 (Oshlack et al.), describes the use of a bittering agent in opioid containing patches to prevent the diversion of opioids by addicts and other recreational drug abusers. According to the publication, the bittering agent is “not releasable when the dosage form is administered intact but which are releasable when the dosage form is broken or tampered with in order to release the opioid from the transdermal system.” See par. 160.

### SUMMARY OF THE INVENTION

The present invention represents a system, with which the noted disadvantages can be avoided. Thus, the present invention operates as a label, which contains a thinning agent that, with the help of endogenous body head, enters into the natural body openings of the upper airways and there leads to a liquefying of the secretions caused by the cold. The invention provides the use of a “bittering agent” for discouraging children from sucking on the label and orally ingesting the active ingredients.

The object on which the present invention is based is thereby resolved by a label, which is characterized in that it can be adhered to clothing worn close to the body and which has a thinning agent, which is released from the adhered label to the surrounding environment of the clothing wearer and enters into the natural body openings of the upper airways and can liquefy accumulated airway secretions caused by the cold.

### DETAILED DESCRIPTION OF THE INVENTION

The present invention may be understood more readily by reference to the following detailed description of preferred embodiments of the invention and the Examples included therein.

Definitions and Use of Terms

As used in this specification and in the claims which follow, the singular forms “a,” “an” and “the” include plural referents unless the context clearly dictates otherwise. Thus, for example, reference to “an ingredient” includes mixtures of ingredients, reference to “an active pharmaceutical agent” includes more than one active pharmaceutical agent, and the like.

“Treating” or “treatment” of a disease includes (1) preventing the disease from occurring in an animal that may be predisposed to the disease but does not yet experience or display symptoms of the disease, (2) inhibiting the disease, i.e. arresting its development, or (3) relieving the disease, i.e. causing regression of the disease.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Therapeutically effective amount” means that amount which, when administered to an animal for treating a disease, is sufficient to effect such treatment for the disease.

Discussion

In a first principal embodiment the invention provides a method of liquefying accumulated airway secretions caused by the common cold comprising: (a) providing an adhesive label that comprises an adhesive layer and an active ingredient layer; and (b) applying the label to the clothing of a human suffering from the common cold; wherein said active ingredient layer comprises a thinning mixture and a bittering agent. In another embodiment the invention provides a label for liquefying accumulated airway secretions caused by the common cold comprising an adhesive layer and an active ingredient layer, wherein said active ingredient layer comprises a thinning mixture and a bittering agent.

The bitterness of a compound can be measured according to section 2.18.5 of the European Pharmacopeia 5.0. As explained therein, the bitterness value of a compound is the reciprocal of the dilution of the compound, a liquid or an extract that still has a bitter taste. It is determined by reference to quinine hydrochloride, the bitterness value of which is set at 200,000. The bitterness value test evaluates the bitterness by testing a test solution in different dilutions through a collective of individuals (organoleptic taste testing). A correction factor for the individual bitterness sensation of the test person is taken into account. In preferred embodiment, the bittering agent has a bitterness value of greater than 10,000, 100,000, or 200,000.

The bitterness of the bittering agent can also be determined by using electronic devices like the eTongue device from Alpha M.O.S. (www.alpha-moss.corn) or the electronic tongue (www.electronictongue.com). The eTongue and Electronic Tongue devices use a set of different electrodes to assess bitterness. The electronic raw signals are processed and the different sensor reactions are revealed by statistical pattern recognition techniques.

In a preferred embodiment, the substance need only be present in an amount of about 1000, 500 or even 200 ppb to be tasted as bitter. An exemplary bittering agent is Bitrex® (denatonium benzoate), available from Macfarlan Smith. Denatonium benzoate is reportedly the most bitter substance known, in which 50-100 ppb is sufficient to be tasted as bitter. Another suitable bittering agent is naringin, the major bioflavonoid in grapefruit and gives grapefruit juice its bitter taste. Naringin is known chemically as 4′,5,7-trihydroxyflavanone-7-rhamoglucoside.

One or more bittering agents should be present in the labels of the present invention in an amount ranging from about 1 to about 1000 ppm per label based on the weight of the thinning agent or the thinning agent combined with the adhesive layer, as described below in greater detail, In more preferred embodiments, the bittering agent is present in an amount ranging from about 1 to about 100 ppm, from about 1 to about 20 ppm, or most preferably about 8 ppm.

Alternatively, the bittering agent can be measured by its total weight in the label, or its weight relative to other ingredients in the formulation. Thus, the bittering agent may be present in the final label in an amount of from about 0.01 to about 1.0 mg, from about 0.05 to about 0.5 mg. or from about 0.1 to about 0.25 mg. Alternatively or in addition, the bittering agent can be present in weight ratio of from about 0.00001:1 to about 0.01:1, or from about 0.0001:1 to about 0.001:1, or from about 0.0002:1 to about 0.0008:1, relative to the weight of the thinning agents (for example, eucalyptus oil and camphor).

The label of the present invention can be characterized by an adhesive layer and/or a layer for sticky adhesion, a removable protective layer for the adhesive layer or the sticky adhesive layer and a non-woven material containing a thinning agent, in particular, a mixture of two or more ether oils as the thinning agent.

In addition, the label of the present invention can be characterized by a thinning agent that can release an initial dose at the beginning of the use and thereafter, a maintenance dose of the thinning agent over a longer time period for liquefying of the accumulated airway secretions caused by the cold, whereby the release rate in milligrams of thinning agent/non-woven layer surface/hour of the initial dose is greater than that of the maintenance dose.

Furthermore, the label of the present invention can be characterized in that the thinning agent can release an initial dose of 100 to 300 mg/hour, preferably of approximately 150 mg/hour, over the first two hours after beginning the use, and thereafter, over at least six hours, a maintenance dose of 10 to 30 mg/hour, preferably of approximately 15 mg/hour, for liquefying accumulated airway secretions caused by a cold.

In addition, the label of the present invention can be characterized in that the adhesive layer can be attained exclusively from the monomers methyl-acrylate, 2-ethyl-hexl-acrylate, and acrylic acid by radical copolymerization with the additional of a cross-linking agent.

Further, the label of the present invention can be characterized in that the adhesive layer can be attained with aluminum acetyl acetonate as the cross-linking agent, in particular, in an amount of 0.04 to 1% (with reference to a weight of all monomers).

Additionally, the label of the present invention can be characterized in that the adhesive layer and the non-woven material layer have been connected to one another when in a wet state and thereafter have been dried.

In addition, the label according to the present invention can be characterized by a synthetic spun mat as the non-woven material layer (carrier mat), in particular, with a coating weight per unit area of 70 to 130 g/m².

Furthermore, the label of the present invention can be characterized by polyester, rayon, Trevira, Dralon, or Modal as the material of the synthetic spun mat.

In addition, the label according to the present invention can be characterized by a fleecy-appearing and/or white or colored non-woven mat layer.

Additionally, the label of the present invention can be characterized by a mixture of ether oils as the thinning agent, wherein the mixture includes an ether oil, which, with a temperature of the clothing worn close to the body, in particular, in the range of 30 to 34° C., does not have a low viscosity or is solid, and the mixture also includes an ether oil, which is fluid with the temperature of the clothing worn close to the body, wherein the mixture of the oils with temperatures of the clothing worn close to the body likewise is fluid, without the need for other assisting materials.

Further, the label according to the present invention can be characterized by a mixture of ether oils of a plant base as the thinning agent, wherein the ether oils are serviceable for secretolysis of airway secretions.

In addition, the label of the present invention can be characterized by a mixture of ether oils from plant components, whose contents or primary contents are selected from a group of terpenes, preferably from a group of monoterpenes, in particular, from the group consisting of 1,8-cineol eucalyptol, camphor, camphene, menthol, aterpinol, thymol, pinene, and limonene.

The label of the present invention can be further characterized in that the thinning agent contains a mixture of eucalyptus oil and camphor as the ether oil, or comprises preferably a weight ratio of eucalyptus oil:camphor of approximately 3:1.

Furthermore, the label of the present invention can be characterized by eucalyptus oil as the thinning agent or as one of its components, preferably in combination with camphor. In a preferred embodiment, the label comprises from about 50 to about 1,000 mg, from about 100 to about 500 mg, or from about 150 to about 250 mg. of eucalyptus oil. When camphor is present, it is preferably present in an amount of from about 10 to about 500, from about 20 to about 250, or from about 50 to about 100 mg per label.

In yet another embodiment, the label comprises pine oil and/or thyme oil, in addition to the eucalyptus, optionally including camphor in the amounts described above. Individual labels may comprise from about 5 to about 300, from about 10 to about 200, or from about 15 to about 75 mg. of pine oil, thyme oil, or a combination thereof.

The label according to the present invention can also be characterized by a size of 20 to 200 cm² and preferably 30 to 60 cm².

In addition, the label of the present invention can be characterized in that one label or multiple labels that are sufficient for an acute cold are found in one package, which preferably is gas impermeable.

The penetration of medications in the natural openings of the body surfaces of the airways is determined substantially by the physical-chemical qualities of the substance. In this regard, the vapor pressure and the temperature of ebullition or the volatility of a substance play a role. Here, it was surprisingly found that the eutectic and self-liquefying mixture of the liquid eucalyptus oil and the solid, crystallized camphor, in a combination of approximately 3:1, enters excellently into the body openings of the airways from the label and in addition, leads to a liquefying of the secretions there. It is no longer necessary to use ether oils in addition co the label. No further vehicle is necessary, such as the turpentine oil, alcohol, Vaseline, etc., used in other topical salve formulations, in order to transport the contents to the airways. It is known that turpentine oil strengthens the liquefying effect; indeed, it was also determined that the migration of the turpentine oil is so incense that is seeps through the packaging means, in which case, the stability and security bases are also detrimentally affected.

Also, by application of the label on the clothing of the body infected by the cold, a contact of the otherwise irrigating ether oils with all mucous membranes (eyes, stomach) and other body surfaces (skin) is avoided, and in spite of that, the airways blocked with secretions are reached. These secretions comprise mucous, which regulate the secretions via disulfide bridges to the protein polymers. It has been shown now that the exclusive separation of the disulfide bridges, for example, by acetyl-cysteine (Fluimucil™. products, Zambon) leads not so certainly to the desired facilitation, such as hydrating of the mucous, which is accomplished by eucalyptus oil as well as by guaifenesine. As a supplement, the camphor works here, which leads to a cold irritation on the mucous membranes and therewith, counteracts the inflammation-indicating heat (hyperemia).

It is important that when ether oils are released in a non-diluted form for inhalation, the release of the oils takes place non-abruptly, because then the ether oils can be released in too concentrated of a form and can lead to a two-phase reverse effect, which can be undesirable. The ether oils should be diluted so highly that the odor is only slightly discernible (Boyd and Sheppard, 1970). This is optimally achieved by the combination of the label (application of the oil in an absorption mat) and the selection of the body temperature of 30-34° C., as the evaporation behavior with in vitro-measurement makes clear. No additional supplements for diluting, such as ethanol, Vaseline, and so on are required. By means of the control by the non-woven material and temperature, emissions in two phases or speeds can be achieved, specifically, a higher rate in the first two hours (initial dose with approximately 150 mg oil/hour) and a lower, so-called maintenance dose (with approximately 15 mg oil/hour) after two hours, which stops after at least six hours, Thus, the label is particularly well suited for use overnight on pajamas.

It is important with the manufacturing of such a label with this type of effective liquefying system that the thinning agent does not engage and liquefy the polymers on the label. It was discovered that the use of a completely specialized acrylate-copolymer in connection with a defined amount of cross-linking agent for antagonizing eventual liquefying of the label leads to a stable product, without using other protective materials, such as inhibiting films, aluminum damping, or the like, makes possible an optimal adhesion on various surfaces of the clothing (natural fibers, such as wool or cotton, as well as synthetic materials, such as polyester, polyamides, etc.). and is removable without residue. A particularly good connection of the adhesion matrix with the absorption mat is achieved, in that in a wet, that is, a solution-retaining state, the connection between both layers is created and is subsequently dried. Then, also no debonding or pulling of filaments can take place by the addition of liquefying ether oils.

As the material for absorbing the ether oils, an absorptive, somewhat thicker non-woven material is suitable. The best are non-woven materials (“nonwovens”) with a coating weight per unit area of at least 70 to 130 g/m², which are manufactured, for example, from types of polyester or rayon. As the protective film for the adhered side of the label, a siliconized polyester film, for example. Hostaphan RN 100 from Diafoil, Noechst, Germany, easy/easy, that is known to the practitioner can be used, which should not be too thin (at least 36 μm layer thickness, preferably 100 μm layer thickness), so that the label can be used well in practice from 30 to 200 cm², preferably, from 30 to 60 cm².

### EXAMPLES

The following examples are put forth so as to provide those of ordinary skill in the art with a complete disclosure and description of how the compounds claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C or is at room temperature, and pressure is at or near atmospheric.

### Example 1

In order to manufacture 100 m² label rolls, one adds 0.051 kg aluminum acetyl acetonate to 28.858 kg of a 35% (m/m) solution of an acrylate-adhesive (Durotak 87-2852. National Starch and Chemical B.V., NL-Zutphen). By stirring every half hour, the solution is homogenized and subsequently spread with a coating knife onto a siliconized, 100 μm thick polyester film (FL 2000 100 μ1-S, Rexam Release B.V. NL-Apeldoorn) in a wet layer thickness of 400 μm. Before drying, it is covered with Paramoll N260/100 (polyester non-woven material of the Fa. Lohmann, D-Andernach) and subsequently dried (15 minutes at 70° C.). The homogenous laminate formed thereby is made into individual labels of 60 cm² by cutting. Directly before packaging in composite packaging material-sealed pouches, the labels are applied by means of a spray nozzle with 500 mg of the ether oil mixture of eucalyptus oil:camphor of 3:1. One obtains a label, which contains an adhesive part of 102 g/m² and 20% eucalyptus oil as well as 6.7% camphor.

### Example 2

In order to make 100 m² rolls of labels, one adds 0.051 kg aluminum acetyl acetonate to 28.858 kg of a 35% (m/m) solution of an acrylate-adhesive (Durotak 87-2852, National Starch and Chemical B.V., NL-Zutphen). By stirring every half hour, the solution is homogenized and subsequently spread with a coating knife onto a siliconized, 100 μm thick polyester film (FL 2000 100 μ1-S, Rexam Release B.V., NL-Apeldoorn) in a wet layer thickness of 400 μm. Before drying, it is covered with TWE-non-woven material 100 (rayon non-woven material of Fa. TWE, D-Emsstaetten) and subsequently dried (15 minutes at 70° C.). The homogeneous laminate that is thereby made is made into individual labels of 59 cm² by stamping. Directly before packaging in composite packaging material-sealed pouches, the labels are applied by means of a spray nozzle with 500 mg of the ether oil mixture of eucalyptus oil:camphor of 3:1. One obtains a label, which contains an adhesive part of 102 g/m² and 20% eucalyptus oil as well as 6.7% camphor.

### Example 3

Representative formulations for a label including a bittering agent are described in Tables 1, 2 and 3. The manufacturing process is identical to the process described in examples 1 and 2, except that an appropriate amount of Bitrex is mixed with thinning agents/essential oils prior to application.

TABLE 1

| | Quantity per | Composition |
|---|---|---|
| Materials | label (mg) | % |
| Eucalyptus Oil, Eu. Ph. | 187.500 | 26.97% |
| Racemic camphor, Eu. Ph. | 62.500 | 8.99% |
| Bitrex | 0.125 | 0.02% |
| Duro-Tak 180-129A | 439.000 | 63.14% |
| Aluminium Triacetylacetonate | 5.488 | 0.79% |
| Ethanol | 0.625 | 0.09% |
| Total | 695.238 | 100.00% |
| 100% viscose support (29.3 cm2) | 351.600 | |
| PET 75 μm (29.3 cm2) silicone | 307.600 | |
| layer | | |
| Total | 1.354,438 | |

TABLE 2

| | Quantity per | Composition |
|---|---|---|
| Materials | label (mg) | % |
| Eucalyptus Oil, Eu. Ph. | 187.500 | 28.73% |
| Pinus sylvestris essential oil, FU XI | 20.000 | 3.06% |
| Bitrex | 0.104 | 0.02% |
| Duro-Tak 180-129A | 439.000 | 67.27% |
| Aluminium Triacetylacetonate | 5.488 | 0.84% |
| Ethanol | 0.519 | 0.08% |
| Total | 652.611 | 100.00% |
| 100% viscose support (29.3 cm2) | 351.600 | |
| PET 75 μm (29.3 cm2) silicone | 307.600 | |
| layer | | |
| Total | 1.311,811 | |

TABLE 3

| | Quantity per | Composition |
|---|---|---|
| Materials | label (mg) | % |
| Eucalyptus Oil, Eu. Ph. | 187.500 | 27.87% |
| Mountain Pine essential oil | 20.000 | 2.97% |
| Thyme Oil, Eu.Ph. | 20.000 | 2.97% |
| Bitrex | 0.114 | 0.02% |
| Duro-Tak 180-129A | 439.000 | 65.26% |
| Aluminium | 5.488 | 0.82% |
| Ethanol | 0.569 | 0.08% |
| Total | 672.671 | 100.00% |
| 100% viscose support (29.3 cm2) | 351.600 | |
| PET 75 μm (29.3 cm2) silicone | 307.600 | |
| layer | | |
| Total | 1.311,811 | |

Throughout this application, various publications are referenced. The disclosures of these publications in their entireties are hereby incorporated by reference into this application in order to more filly describe the state of the art to which this invention pertains. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of liquefying accumulated airway secretions caused by the common cold comprising:
a) providing an adhesive label that comprises an adhesive layer and an active ingredient layer; and
b) applying the label to the clothing of human suffering from the common cold; wherein
said active ingredient layer comprises a thinning mixture and a bittering agents,
said active ingredient layer comprises a non-woven fabric impregnated with said thinning mixture,
said thinning mixture comprises eucalyptus oil and at least one further thinning agent and
said bittering agent comprises denatonium benzoate in a weight ratio of 0.00001 to 0.01 relative to the weight of the thinning agents.

2. (canceled)

3. (canceled)

4. The method of claim 1 wherein said thinning mixture comprises eucalyptus oil and a volatile thinning agent.

5. The method of claim 1 wherein said active ingredient layer comprises:
a) from about 0.05 to about 05 mg. of denatonium benzoate;
b) from about 10 to about 500 mg. of eucalyptus oil; and
c) from about 20 to about 250 mg. of camphor oil.

6. The method of claim 1 wherein said active ingredient layer comprises:
a) from about 0.05 to about 05. mg. of denatonium benzoate;
b) from about 10 to about 500 mg. of eucalyptus oil; and
c) from about 10 to about 200 mg. of thyme oil or pine oil or a combination thereof.

7. The method of claim 1 wherein said adhesive label releases from about 100 to about 300 mg/hr of said thinning mixture during hours 1 and 2 after step (b).

8. The method of claim 1 wherein said adhesive label releases from about 100 to about 300 mg/hr of said thinning mixture during hours 1 and 2 after step (b).

9. A label for liquefying accumulated airway secretions caused by the common cold comprising an adhesive layer and an active ingredient layer, wherein
said active ingredient layer comprises a thinning mixture and a bittering agent,
said active ingredient layer comprises a non-woven fabric impregnated with said thinning mixture,
said thinning mixture comprises eucalyptus oil and at least one further thinning agent and
said bittering agent comprises denatonium benzoate in a weight ratio of 0.00001 to 0.01 relative to the weight of the thinnings agents.
said active ingredient layer comprise a thinning mixture and a bittering agent.

10. (canceled)

11. (canceled)

12. The label of claim 9 wherein said active ingredient layer comprises:
a) from about 0.05 to about 0.5 mg. of denatonium benzoate;
b) from about 10 to about 500 mg. of eucalyptus oil; and
c) from about 20 to about 250 mg. of camphor oil.

13. The label of claim 9 wherein said active ingredient layer comprises:
a) from about 0.05 to about 0.5 mg. of denatonium benzoate;
b) from about 10 to about 500 mg. of eucalyptus oil; and
c) from about 10 to about 200 mg. of thyme oil or pine oil or a combination thereof.

14. The label of claim 9, wherein said adhesive layer is adjacent to said active ingredient layer.

15. The label of claim 9, wherein said adhesive label further comprises a removable protective layer adjacent to said adhesive layer.

16. The label of claim 9 wherein said active ingredient layer comprise a non-woven fabric impregnated with said thinning mixture.

17. The label of claim 9 wherein said adhesive layer comprises a copolymer of methyl-acrylate, 2-ethyl-hexl-acrylate, and acrylic acid, and a cross-linking agent.

18. A method of making the label of claim 16 comprising contacting said non-woven fabric to said adhesive layer when said adhesive layer is in a wet state.

19. The label of claim 16 wherein said non-woven fabric comprises a synthetic spun mat with a coating weight per unit area of 70 to 130 g/m².

20. The label of claim 9 wherein said label has an area of from about 20 to about 200 cm².
